SEQUENCE LISTING (63 sequences)

SEQ ID NO: 1 - protein, 143 aa, synthetic construct
MRKLRRLKRKIAHKVKKYFKLSQRSKDRLVGVHPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 2 - protein, 142 aa, synthetic construct
MRKLRRLKRKIAHKVKKYFKLSQRXXDRXXXVHPDXVKVXHRALELTPVDFGITEGVRSLETQKKYVAEGKXKTMKSRHLHGLAVDVVAYPKDKDTWNMKXYRMXADXFKQAGRELGVSVEXXXXVSXXXGVHFQLPHSKYP

SEQ ID NO: 3 - protein, 143 aa, synthetic construct
MRKLRRLKRKIAHKVKKYFKLSQRSKDRLVGVHPDLVKVVHRALELTPVDFGITEGVRSLETQKKYVAEGKXKTMKSRHLHGLAVDVVAYPKDKDTWNMKYYRMIADAFKQAGRELGVSVEXXGDXVSXKXGVHFQLPHSKYP

SEQ ID NO: 4 - protein, 143 aa, synthetic construct
MRKLRRLKRKIAHKVKKYFKLSQRSKDRXVGVHPDXVKVVHRALELTPVDFGITEGVRSLETQKKYVAEGKSKTMKSRHLHGLAVDVVAYPKDKDTWNMKYYRMIADAFKQAGRELGVSVEWGXDWVSFKDGVHFQLPHSKYP

SEQ ID NO: 5 - protein, 143 aa, synthetic construct
MRKLRRLKRKIAHKVKKYFKLSQRSKDRLVGVHPDLVKVXHRALELTPVDFGITEGVRSLETQKKYVAEGKSKTMKSRHLHGLAVDVVAYPKDKDTWNMKYYRMIADAFKQAGRELGVSVEWGGXWVSFKDGVHFQLPHSKYP

SEQ ID NO: 6 - protein, 142 aa, synthetic construct
MRKLRRLKRKIAHKVKKYFKLSQRXKDRLVGVHPDLVKXVHRALELTPVDFGITEGVRSLETQKKYVAEGKSKTMKSRHLHGLAVDVVAYPKDKDTWNMKXYRMXADXKQAGRELGVSVEWGGDWVSFKDGVHFQLPHSKYP

SEQ ID NO: 7 - protein, 143 aa, synthetic construct
MRKLRRLKRKIAHKVKKYFKLSQRSXDRLXXVHPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFXDGVHFQLPHSKYP

SEQ ID NO: 8 - protein, 29 aa, synthetic construct
RGLRRLGRKIAHGVKKYGPTVLRIIRIAG

SEQ ID NO: 9 - protein, 143 aa, synthetic construct
MRKLRRLKRKIAHKVKKYFKLSQRXXXXXXXXXXXXXXXXHRALELTPVDFGITEGVRSLETQKKYVXXXXXXTMKSRHLHGLAVDVVAYPKDKDTXXXXXXXXXXXXXXQAGRELGVXXXXXXXXXXXXXGVHFQLXXXXXX

SEQ ID NO: 10 - protein, 143 aa, synthetic construct
MRKLRRLKRKIAHKVKKYFKLSQRSCDRLVGVHPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 11 - protein, 143 aa, synthetic construct
MRKLRRLKRKIAHKVKKYFKLSQRSIDRLVGVHPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 12 - protein, 143 aa, synthetic construct
MRKLRRLKRKIAHKVKKYFKLSQRSYDRLVGVHPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 13 - protein, 143 aa, synthetic construct
MRKLRRLKRKIAHKVKKYFKLSQRSADRLVGVHPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 14 - protein, 143 aa, synthetic construct
MRKLRRLKRKIAHKVKKYFKLSQRSPDRLVGVHPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 15 - protein, 143 aa, synthetic construct
MRKLRRLKRKIAHKVKKYFKLSQRSRDRLVGVHPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 16 - protein, 143 aa, synthetic construct
MRKLRRLKRKIAHKVKKYFKLSQRSDDRLVGVHPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 17 - protein, 143 aa, synthetic construct
MRKLRRLKRKIAHKVKKYFKLSQRSSDRLVGVHPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 18 - protein, 143 aa, synthetic construct
MRKLRRLKRKIAHKVKKYFKLSQRSNDRLVGVHPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 19 - protein, 143 aa, synthetic construct
MRKLRRLKRKIAHKVKKYFKLSQRSKDRLVGVAPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 20 - protein, 143 aa, synthetic construct
MRKLRRLKRKIAHKVKKYFKLSQRSKDRLVGVRPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 21 - protein, 143 aa, synthetic construct
MRKLRRLKRKIAHKVKKYFKLSQRSKDRLVGVNPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 22 - protein, 143 aa, synthetic construct
MRKLRRLKRKIAHKVKKYFKLSQRSKDRLVGVDPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 23 - protein, 143 aa, synthetic construct
MRKLRRLKRKIAHKVKKYFKLSQRSKDRLVGVCPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 24 - protein, 143 aa, synthetic construct
MRKLRRLKRKIAHKVKKYFKLSQRSKDRLVGVQPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 25 - protein, 143 aa, synthetic construct
MRKLRRLKRKIAHKVKKYFKLSQRSKDRLVGVEPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 26 - protein, 143 aa, synthetic construct
MRKLRRLKRKIAHKVKKYFKLSQRSKDRLVGVGPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 27 - protein, 143 aa, synthetic construct
MRKLRRLKRKIAHKVKKYFKLSQRSKDRLVGVIPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 28 - protein, 143 aa, synthetic construct
MRKLRRLKRKIAHKVKKYFKLSQRSKDRLVGVLPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 29 - protein, 143 aa, synthetic construct
MRKLRRLKRKIAHKVKKYFKLSQRSKDRLVGVKPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 30 - protein, 143 aa, synthetic construct
MRKLRRLKRKIAHKVKKYFKLSQRSKDRLVGVMPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 31 - protein, 143 aa, synthetic construct
MRKLRRLKRKIAHKVKKYFKLSQRSKDRLVGVFPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 32 - protein, 143 aa, synthetic construct
MRKLRRLKRKIAHKVKKYFKLSQRSKDRLVGVPPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 33 - protein, 143 aa, synthetic construct
MRKLRRLKRKIAHKVKKYFKLSQRSKDRLVGVSPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 34 - protein, 143 aa, synthetic construct
MRKLRRLKRKIAHKVKKYFKLSQRSKDRLVGVTPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 35 - protein, 143 aa, synthetic construct
MRKLRRLKRKIAHKVKKYFKLSQRSKDRLVGVWPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 36 - protein, 143 aa, synthetic construct
MRKLRRLKRKIAHKVKKYFKLSQRSKDRLVGVYPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 37 - protein, 143 aa, synthetic construct
MRKLRRLKRKIAHKVKKYFKLSQRSKDRLVGVVPDLVKVVHRALELTPVDFGITEGVRSLETQKKYVAEGKSKTMKSRHLHGLAVDVVAYPKDKDTWNMKYYRMIADAFKQAGRELGVSVEWGGDWVSFKDGVHFQLPHSKYP

SEQ ID NO: 38 - protein, 142 aa, synthetic construct
MRKLRRLKRKIAHKVKKYFKLSQRSKDRLVGVHPDLVKVVHRALELTPVDFGITEGVRSLETQKKYVAEGKSKTMKSRHLHGLAVDVVAYPKDKDTWNMKYYRMIADAFKQAGRELGVSGEWGGDWVSFKDGVHFQLPHSKY

SEQ ID NO: 39 - protein, 142 aa, synthetic construct
MRKLRRLKRKIAHKVKKYFKLSQRSKDRLVGVHPDLVKVVHRALELTPVDFGITEGVRSLETQKKYVAEGKSKTMKSRHLHGLAVDVVAYPKDKDTWNMKYYRMIADAFKQAGRELGVSAEWGGDWVSFKDGVHFQLPHSKY

SEQ ID NO: 40 - protein, 142 aa, synthetic construct
MRKLRRLKRKIAHKVKKYFKLSQRSKDRLVGVHPDLVKVVHRALELTPVDFGITEGVRSLETQKKYVAEGKSKTMKSRHLHGLAVDVVAYPKDKDTWNMKYYRMIADAFKQAGRELGVSLEWGGDWVSFKDGVHFQLPHSKY

SEQ ID NO: 41 - protein, 142 aa, synthetic construct
MRKLRRLKRKIAHKVKKYFKLSQRSKDRLVGVHPDLVKVVHRALELTPVDFGITEGVRSLETQKKYVAEGKSKTMKSRHLHGLAVDVVAYPKDKDTWNMKYYRMIADAFKQAGRELGVSIEWGGDWVSFKDGVHFQLPHSKY

SEQ ID NO: 42 - protein, 142 aa, synthetic construct
MRKLRRLKRKIAHKVKKYFKLSQRSKDRLVGVHPDLVKVVHRALELTPVDFGITEGVRSLETQKKYVAEGKSKTMKSRHLHGLAVDVVAYPKDKDTWNMKYYRMIADAFKQAGRELGVSPEWGGDWVSFKDGVHFQLPHSKY

SEQ ID NO: 43 - protein, 142 aa, synthetic construct
MRKLRRLKRKIAHKVKKYFKLSQRSKDRLVGVHPDLVKVVHRALELTPVDFGITEGVRSLETQKKYVAEGKSKTMKSRHLHGLAVDVVAYPKDKDTWNMKYYRMIADAFKQAGRELGVSFEWGGDWVSFKDGVHFQLPHSKY

SEQ ID NO: 44 - protein, 142 aa, synthetic construct
MRKLRRLKRKIAHKVKKYFKLSQRSKDRLVGVHPDLVKVVHRALELTPVDFGITEGVRSLETQKKYVAEGKSKTMKSRHLHGLAVDVVAYPKDKDTWNMKYYRMIADAFKQAGRELGVSMEWGGDWVSFKDGVHFQLPHSKY

SEQ ID NO: 45 - protein, 142 aa, synthetic construct
MRKLRRLKRKIAHKVKKYFKLSQRSKDRLVGVHPDLVKVVHRALELTPVDFGITEGVRSLETQKKYVAEGKSKTMKSRHLHGLAVDVVAYPKDKDTWNMKYYRMIADAFKQAGRELGVSWEWGGDWVSFKDGVHFQLPHSKY

SEQ ID NO: 46 - protein, 142 aa, synthetic construct
MRKLRRLKRKIAHKVKKYFKLSQRSKDRLVGVHPDLVKVVHRALELTPVDFGITEGVRSLETQKKYVAEGKSKTMKSRHLHGLAVDVVAYPKDKDTWNMKYYRMIADAFKQAGRELGVSREWGGDWVSFKDGVHFQLPHSKY

SEQ ID NO: 47 - protein, 142 aa, synthetic construct
MRKLRRLKRKIAHKVKKYFKLSQRSKDRLVGVHPDLVKVVHRALELTPVDFGITEGVRSLETQKKYVAEGKSKTMKSRHLHGLAVDVVAYPKDKDTWNMKYYRMIADAFKQAGRELGVSNEWGGDWVSFKDGVHFQLPHSKY

SEQ ID NO: 48 - protein, 142 aa, synthetic construct
MRKLRRLKRKIAHKVKKYFKLSQRSKDRLVGVHPDLVKVVHRALELTPVDFGITEGVRSLETQKKYVAEGKSKTMKSRHLHGLAVDVVAYPKDKDTWNMKYYRMIADAFKQAGRELGVSDEWGGDWVSFKDGVHFQLPHSKY

SEQ ID NO: 49 - protein, 142 aa, synthetic construct
MRKLRRLKRKIAHKVKKYFKLSQRSKDRLVGVHPDLVKVVHRALELTPVDFGITEGVRSLETQKKYVAEGKSKTMKSRHLHGLAVDVVAYPKDKDTWNMKYYRMIADAFKQAGRELGVSCEWGGDWVSFKDGVHFQLPHSKY

SEQ ID NO: 50 - protein, 142 aa, synthetic construct
MRKLRRLKRKIAHKVKKYFKLSQRSKDRLVGVHPDLVKVVHRALELTPVDFGITEGVRSLETQKKYVAEGKSKTMKSRHLHGLAVDVVAYPKDKDTWNMKYYRMIADAFKQAGRELGVSQEWGGDWVSFKDGVHFQLPHSKY

SEQ ID NO: 51 - protein, 142 aa, synthetic construct
MRKLRRLKRKIAHKVKKYFKLSQRSKDRLVGVHPDLVKVVHRALELTPVDFGITEGVRSLETQKKYVAEGKSKTMKSRHLHGLAVDVVAYPKDKDTWNMKYYRMIADAFKQAGRELGVSEEWGGDWVSFKDGVHFQLPHSKY

SEQ ID NO: 52 - protein, 142 aa, synthetic construct
MRKLRRLKRKIAHKVKKYFKLSQRSKDRLVGVHPDLVKVVHRALELTPVDFGITEGVRSLETQKKYVAEGKSKTMKSRHLHGLAVDVVAYPKDKDTWNMKYYRMIADAFKQAGRELGVSHEWGGDWVSFKDGVHFQLPHSKY

SEQ ID NO: 53 - protein, 142 aa, synthetic construct
MRKLRRLKRKIAHKVKKYFKLSQRSKDRLVGVHPDLVKVVHRALELTPVDFGITEGVRSLETQKKYVAEGKSKTMKSRHLHGLAVDVVAYPKDKDTWNMKYYRMIADAFKQAGRELGVSKEWGGDWVSFKDGVHFQLPHSKY

SEQ ID NO: 54 - protein, 142 aa, synthetic construct
MRKLRRLKRKIAHKVKKYFKLSQRSKDRLVGVHPDLVKVVHRALELTPVDFGITEGVRSLETQKKYVAEGKSKTMKSRHLHGLAVDVVAYPKDKDTWNMKYYRMIADAFKQAGRELGVSSEWGGDWVSFKDGVHFQLPHSKY

SEQ ID NO: 55 - protein, 142 aa, synthetic construct
MRKLRRLKRKIAHKVKKYFKLSQRSKDRLVGVHPDLVKVVHRALELTPVDFGITEGVRSLETQKKYVAEGKSKTMKSRHLHGLAVDVVAYPKDKDTWNMKYYRMIADAFKQAGRELGVSTEWGGDWVSFKDGVHFQLPHSKY

SEQ ID NO: 56 - protein, 142 aa, synthetic construct
MRKLRRLKRKIAHKVKKYFKLSQRSKDRLVGVHPDLVKVVHRALELTPVDFGITEGVRSLETQKKYVAEGKSKTMKSRHLHGLAVDVVAYPKDKDTWNMKYYRMIADAFKQAGRELGVSYEWGGDWVSFKDGVHFQLPHSKY

SEQ ID NO: 57 - DNA, 104 bp, synthetic construct
  source 1..104: mol_type = other DNA
atgaattgtatcttgtccggcagaaaattggttatttttattattccgctctgcaccccatttgcattatatagagtatggataagaaaggagtgaaggttatg

SEQ ID NO: 58 - DNA, 138 bp, synthetic construct
  source 1..138: mol_type = other DNA
acgctctgctttttcttattttccaagcatatgatgaatatatagacgttcacccacaccaagtggggcacgggtacatatgttgttaaggactaaagtcaaataccctataattctagaaaggaggtggccggcatg

SEQ ID NO: 59 - DNA, 75 bp, synthetic construct
  source 1..75: mol_type = other DNA
aagtttaaatatttggattttttaaataaagcgtttacaatatatgtagaaacaacaaagggggagatttgtatg

SEQ ID NO: 60 - protein, 11 aa, synthetic construct
GGGEAAAKGGG

SEQ ID NO: 61 - DNA, 480 bp, synthetic construct
  source 1..480: mol_type = other DNA
atgagctgcggaaaaacccatggccgacatgagaactgtgtatgcgatgcagtggaaaagattttagcagagcaggaggcagttgaagaacagtgtccgactggctgctataccaaccttttaaaccctacgattgctggaaaagacacaattccgtttctcgtttttgataaaaaaggcggattgttctccacattcggaaacgtagggggatttgtggatgatatgcaatgctttgaatccattttcttccgcgtcgaaaaattatgcgattgctgtgcaacactgtctattttacgcccggtcgatgtcaaaggcgataccttaagtgtttgccacccttgcgacccggatttcttcgggctagaaaaaacagatttctgcattgaagtggatctcggatgcttctgcgcgattcagtgcctgtcaccagagctagttgacagaacatcgcctcacaaagataaaaagcatcatcac

SEQ ID NO: 62 - DNA, 429 bp, synthetic construct
  source 1..429: mol_type = other DNA
atgcgtaagctgcgtcgcttgaaacgcaagatcgctcacaaagttaagaagtatttcaagttaagtcagcgcagtaaagatcgtttggtcggtgtccatcctgacctggtaaaggtcgtccaccgtgcccttgaactgaccccagtcgactttgggatcaccgagggggtccgttcattagagacacaaaagaaatatgttgccgagggtaagagcaagacaatgaagagccgccacttacatggattagctgttgacgtcgtggcttatcctaaggataaagacacctggaatatgaaatactaccgcatgattgccgatgcgtttaagcaagcaggccgtgaattaggagtatcagtcgagtggggtggtgactgggtatcatttaaggacggtgtccacttccagttgccgcactccaagtatccc

SEQ ID NO: 63 - DNA, 429 bp, synthetic construct
  source 1..429: mol_type = other DNA
gggatacttggagtgcggcaactggaagtggacaccgtccttaaatgatacccagtcaccaccccactcgactgatactcctaattcacggcctgcttgcttaaacgcatcggcaatcatgcggtagtatttcatattccaggtgtctttatccttaggataagccacgacgtcaacagctaatccatgtaagtggcggctcttcattgtcttgctcttaccctcggcaacatatttcttttgtgtctctaatgaacggaccccctcggtgatcccaaagtcgactggggtcagttcaagggcacggtggacgacctttaccaggtcaggatggacaccgaccaaacgatctttactgcgctgacttaacttgaaatacttcttaactttgtgagcgatcttgcgtttcaagcgacgcagcttacgcat

(19) United States
(12) Patent Application Publication
(10) Pub. No.: US 2025/0324979 A1
(43) Pub. Date: Oct. 23, 2025

(54) LysECD7 VARIANTS AND METHODS OF USING THE SAME

(71) Applicants: Alexandre ISMAIL, Brooklyn, NY (US); Vera PISAREVA, Brooklyn, NY (US); German SABIO, Brooklyn, NY (US); Ellen JORGENSEN, Brooklyn, NY (US); Vishaal BHUYAN, Brooklyn, NY (US); Aanika Biosciences, Inc., Brooklyn, NY (US)

(72) Inventors: Alexandre Ismail, Brooklyn, NY (US); Vera Pisareva, Brooklyn, NY (US); German Sabio, Brooklyn, NJ (US); Ellen Jorgensen, Brooklyn, NY (US); Vishaal Bhuyan, Brooklyn, NY (US)

(21) Appl. No.: 18/689,046

(22) PCT Filed: Sep. 6, 2022
(86) PCT No.: PCT/US2022/042667

§ 371 Date: Mar. 4, 2024

Related U.S. Application Data
(60) Provisional application No. 63/240,365, filed on Sep. 3, 2021.
(60) Provisional application No. 63/273,170, filed on Oct. 29, 2021.

(51) Int. Cl. A01N 63/50; A61K 38/00; C07K 14/32; C12N 9/48; C12N 11/16

(52) U.S. Cl. CPC A01N 63/50; C07K 14/32; C12N 9/48; C12N 11/16; A61K 38/00; C07K 2319/00

(57) ABSTRACT

The present disclosure relates to a group of Antimicrobial peptides (referred to as “LysECD7 variants” or “LysECD7 variant proteins”) that are based on the wild-type enzyme LysECD7 but with specific amino acid substitutions, poly nucleotides and vectors encoding the same, peptide fragments thereof, cells or spores expressing the poly nucleotide or vector, and methods of using and making the same. In addition, the present disclosure relates to spore-associated or microbe-associated LysECD7 variants. The association of LysECD7 variants with spores or microbes can include surface display by covalent or noncovalent association or by mixing. The present disclosure is further directed to fragments or subsequences of LysECD7 variant nucleic acids comprising hybridizable portions of the LysECD7 variant sequence which have use. e.g. in nucleic acid-based assays.

### FIELD OF THE INVENTION

The present disclosure relates generally to the field of LysECD7 variants, spores with surface displayed anti-microbial peptides such as the LysECD7 variants, and methods of using the same.

### BACKGROUND OF THE INVENTION

Antibiotic-resistant microbes (AMRs) are one of the largest threats to human health in the world (1). The widespread use of antibiotics in agriculture has exacerbated the problem. Without effective antibiotics, even the simplest surgical operation or a single scratch could become potentially life-threatening. The United Nations recently released a report stating that the current yearly death toll from drug-resistant diseases is 700,000 and that if no action is taken this could rise to 10 million deaths per year by 2030 and force up to 24 million people into extreme poverty (2). Discovery and development of new antibiotics has slowed considerably in the years following the ‘golden age’ of antibiotic discovery which lasted from the 1940s through the 1970s (3). From 2004-2014 less than one new antibiotic was approved for use per year, and the search for alternative antimicrobial therapies has intensified.

Antimicrobial peptides (AMPs) are a distinct class of molecules found in virtually all forms of life (4). Their action may be specific or against broad classes of microbes. Bacteria and fungi produce antimicrobial peptides to eliminate competition for resources. Plants lack an adaptive immune system and so AMPs form an important component of their immune defence (4). LysECD7 is an AMP and part of a group of bacteriophage lysins that show great promise as alternatives to antibiotics (5). It is derived from the Myoviridae bacteriophage family and has demonstrated activity against a panel of Gram-negative clinical bacterial isolates which included P. aeruginosa, A. baumannii, K. pneumoniae, and Salmonella typhi strains, and against multiresistant strains of E. coli (6). The addition of the N-terminus sheep myeloid peptide of 29 amino acids (SMAP-29) to LysECD7 broadens the range of antimicrobial activity to include not only gram negative but also gram positive bacteria, and enhances stability under different test conditions (8). The sequence of the LysECD7 protein with added SMAP-29 (LysECD7-SMAP) is shown in FIG. 1.

Recently there has been a great deal of interest in medical uses of antimicrobial peptides to combat infection and also biofilm formation on implants (7). They have also been proposed as food preservatives (9,10). Different uses of antimicrobial peptides may call for different levels of antimicrobial effectiveness and different microbe target specificities. Thus, there is a need for additional AMPs.

### SUMMARY OF THE INVENTION

The present disclosure relates to a group of AMPs (referred to as “LysECD7 variants” or “LysECD7 variant proteins”) that are based on the wild-type enzyme LysECD7 but with specific amino acid substitutions, polynucleotides and vectors encoding the same, peptide fragments thereof, cells or spores expressing the polynucleotide or vector, and methods of using and making the same.

The present disclosure further pertains to such variants with a SMAP peptide (RGLRRLGRKIAHGVKKYGPTVLRIIRIAG (SEQ ID NO: 8)) attached to the N or C terminal region of LysECD7 variants. In all parts of this document, the term “LysECD7 variants” or “LysECD7 variant proteins” will include LysECD7 variants and LysECD7 variants conjugated to SMAP.

In addition, the present disclosure relates to spore-associated or microbe-associated LysECD7 variants. The association of LysECD7 variants with spores or microbes can include surface display by covalent or noncovalent association or by mixing. The association of LysECD7 variants with spores or microbes can include expression by way of fusion to a crust protein such as CotY. The spores can further comprises a barcode region and also have a genome modified so that the spore is non-germinating, as described in WO2020243730A1, which is hereby incorporated by reference in its entirety.

The present disclosure is further directed to fragments or subsequences of LysECD7 variant nucleic acids comprising hybridizable portions of the LysECD7 variant sequence which have use, e.g. in nucleic acid-based assays.

### DRAWINGS

FIG. 1 shows the wild-type amino acid sequence, nucleic acid sequence encoding the amino acid sequence, and a nucleic acid sequence that can hybridize therewith.

FIG. 2 is a theoretical, three dimensional model of the LysECD7 protein based upon the crystal structure of ChiXa whose amino acid sequence is 49% homologous with LysECD7.

FIG. 3 is a more detailed view of a theoretical, three-dimensional model of the LysECD7 protein that is color coded to show where the active sites are (indicated by a star) and proposed zones of mutation.

FIG. 4 depicts which amino acid positions showed either a significant increase or decrease in lysis activity when compared to the ‘wild type’ LysECD7-SMAP.

FIG. 5 depicts a vector map of the LysECD7 variant-CotY fusion protein. As depicted, the vector utilizes the pBS1C-backbone of the Bartel reference (see Example section for full citation), which carries a cat gene mediating chloramphenicol resistance in B. subtilis flanked by homologous integration sites for the amyE locus in the genome of B. subtilis, as well as the bla gene for an ampicillin resistance and an origin of replication (ori) for vector propagation in Escherichia coli. The expression cassette is located between the restriction sites EcoRI and PstI. It contains the strongest crust operon promoter PcotYZ, the LysECD7 variant, one of the crust genes (cotV, cotW, cotX, cotY, cotZ, or cgeA), and a transcriptional terminator. The enzymes of use are XbaI (X), NgoMIV (N), AgeI (A), and SpeI (S), where N and A produce compatible overhangs, following the BioBrick RFC2520 cloning standard for fusion proteins, but including a ribosome binding site (RBS) for B. subtilis.

### DETAILED DESCRIPTION

LysECD7 variants as well as fragments or derivatives thereof can comprise or consist of an amino acid sequence with one or more mutations within one or more of the regions selected from amino acid residues 25-40, amino acid residues 68-73, amino acid residues 97-110, and amino acid residues 119-131 of SEQ ID NO: 1 which is the sequence of wild type variant LyseECD7. Other LysECD7 variant embodiments include the afore-mentioned LysECD7 variants attached to SMAP-29. The various variant sequences contemplated by the present disclosure are shown in Table 1 below.

TABLE 1

| | Sequence |
|---|---|
| Variant 1 | MRKLRRLKRKIAHKVKKYFKLSQRXXXXXXXXXXXXXXXXHRALELT |
| | PVDFGITEGVRSLETQKKYVXXXXXXTMKSRHLHGLAVDVVAYPKDKD |
| | TXXXXXXXXXXXXXXQAGRELGVXXXXXXXXXXXXXGVHFQLXXXX |
| | XX (SEQ ID NO: 9) |
| Variant 2 | MRKLRRLKRKIAHKVKKYFKLSQRXXDRXXXVHPDXVKVXHRALELTP |
| | VDFGITEGVRSLETQKKYVAEGKXKTMKSRHLHGLAVDVVAYPKDKDT |
| | WNMKXYRMXADXFKQAGRELGVSVEXXXXVSXXXGVHFQLPHSKYP |
| | (SEQ ID NO: 2) |
| Variant 3 | MRKLRRLKRKIAHKVKKYFKLSQRSKDRLVGVHPDLVKVVHRALELTP |
| | VDFGITEGVRSLETQKKYVAEGKXKTMKSRHLHGLAVDVVAYPKDKDT |
| | WNMKYYRMIADAFKQAGRELGVSVEXXGDXVSXKXGVHFQLPHSKYP |
| | (SEQ ID NO: 3) |
| Variant 4 | MRKLRRLKRKIAHKVKKYFKLSQRSKDRXVGVHPDXVKVVHRALELTP |
| | VDFGITEGVRSLETQKKYVAEGKSKTMKSRHLHGLAVDVVAYPKDKDT |
| | WNMKYYRMIADAFKQAGRELGVSVEWGXDWVSFKDGVHFQLPHSKYP |
| | (SEQ ID NO: 4) |
| Variant 5 | MRKLRRLKRKIAHKVKKYFKLSQRSKDRLVGVHPDLVKVXHRALEL TP |
| | VDFGITEGVRSLETQKKYVAEGKSKTMKSRHLHGLAVDVVAYPKDKDT |
| | WNMKYYRMIADAFKQAGRELGVSVEWGGXWVSFKDGVHFQLPHSKYP |
| | (SEQ ID NO: 5) |
| Variant 6 | MRKLRRLKRKIAHKVKKYFKLSQRXKDRLVGVHPDL VKXVHRALELTP |
| | VDFGITEGVRSLETQKKYVAEGKSKTMKSRHLHGLAVDVVAYPKDKDT |
| | WNMKXYRMXADXKQAGRELGVSVEWGGDWVSFKDGVHFQLPHSKYP |
| | (SEQ ID NO: 6) |
| Variant 7 | MRKLRRLKRKIAHKVKKYFKLSQRSXDRLXXVHPDLVKVVHRALELTP |
| | VDFGITEGVRSLETQKKYVAEGKSKTMKSRHLHGLAVDVVAYPKDKDT |
| | WNMKYYRMIADAFKQAGRELGVSVEWGGDWVSFXDGVHFQLPHSKYP |
| | (SEQ ID NO: 7) |

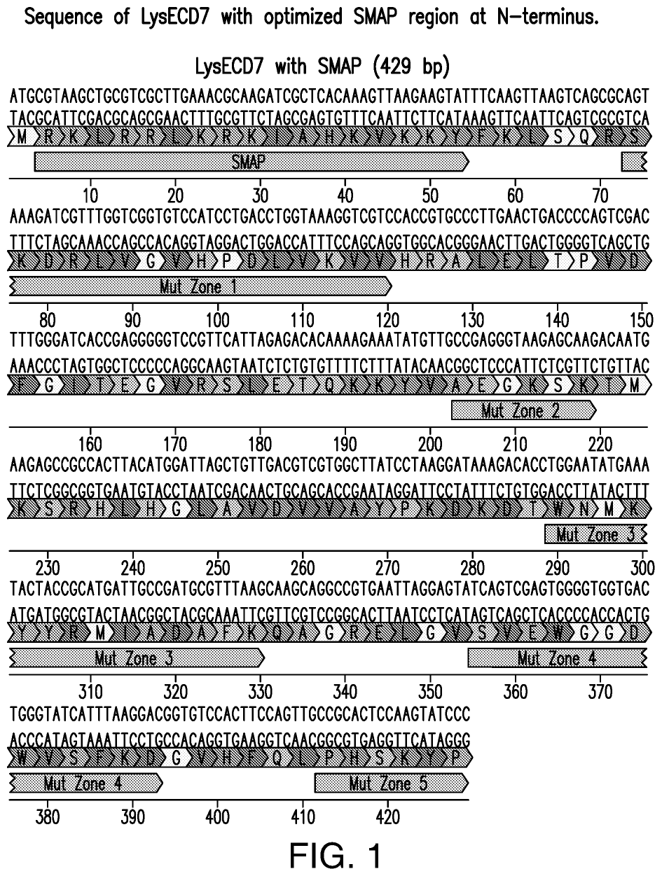

The grayed amino acid residues in the above table of sequences are the possible sites of mutation, signified by an X. The Xs can be any amino acid that is the same as or different from the corresponding amino acid in the wild type sequence as shown in FIG. 1, wherein at least one X is different from that of the wild type sequence. In other words, the Xs of SEQ ID NO: 2, 3, 4, 5, 6, 7, or 9 are selected from the following amino acids: A, R, N, D, C, Q, E, G, H, I, L, K, M, F, P, S, T, W, Y, and V such that at least one X within a given sequence is not the same amino acid as that in the same position in SEQ ID NO: 1. In some embodiments, only one position is mutated. In some embodiments, only two or three positions are mutated. These variants have altered function such as greater or lesser microbial lysis efficiency or altered target specificity.

Table 2 below is a table showing what the amino acid substitutions can be when the wild-type residue is a certain type. For example, according to the 1^{st} row in Table 2, in some embodiments, when an X in any one of SEQ ID Nos: 2 to 7 corresponds to an L in that same position in the wild-type residue of SEQ ID NO: 1, then the amino acid at the X is an amino acid selected from G, A, V, I, P, F, M, and W.

TABLE 2

| Wild Type Amino Acid at | |
|---|---|
| Corresponding X Position | Substitutions |
| L | G, A, V, I, P, F, M, or W |
| V | G, A, L, I, P, F, M, or W |
| W | G, A, V, L, I, P, F, or M |
| G | A, V, L, I, P, F, M, or W |
| A | G, V, L, I, P, F, M, or W |
| I | G, A, V, L, P, F, M, or W |
| F | G, A, V, L, I, P, M, or W |
| F | W or Y |
| S | C, T, Y, N, or Q |
| S | T |
| Y | S, C, T, N, or Q |
| Y | F or W |
| D | E |
| K | R or H |
| K | I, R, D, A, S, C, Y, P, or N |
| R | K or H |
| H | R or K |
| W | Y or F |

Other embodiments include peptides according to the above but also with 1, 2, 3, 4, 5, 6, 7, 8, 9, 10, 11, 12, 13, 14, or 15 conservative substitutions at residues not within the range of 25-40, 68-73, 97-110, 119-131, and 138-143. These ranges are believed to be the regions that participate in binding, and those outside of these ranges can tolerate some conservative alterations without altering the function of the peptide. A conservative substitution is one where the substituted residue is one of similar polarity. For example, the nonpolar (hydrophobic) amino acids—alanine, leucine, isoleucine, valine, proline, phenylalanine, tryptophan and methionine—can be substituted for each other. Similarly, the polar neutral amino acids—glycine, serine, threonine, cysteine, tyrosine, asparagine, and glutamine—can be substituted for each other. The positively charged (basic) amino acids-arginine, lysine and histidine—can be substituted for each other. The negatively charged (acidic) amino acids-aspartic acid and glutamic acid—can be substituted for each other.

In some embodiments, the LysECD7 variant is one that has a substitution at position 26 (K) of the wild-type sequence wherein the substitution is selected from the following group of amino acids: I, R, D, A, S, C, Y, P, and N. Such sequences are shown below in Table 3. These peptides exhibit a higher lysis activity than the wild-type form.

TABLE 3

| | Sequence |
|---|---|
| Variant 8a | MRKLRRLKRKIAHKVKKYFKLSQRSCDRLVGVHPDLVKVVHRALELTP |
| | VDFGITEGVRSLETQKKYVAEGKSKTMKSRHLHGLAVDVVAYPKDKDT |
| | WNMKYYRMIADAFKQAGRELGVSVEWGGDWVSFKDGVHFQLPHSKYP |
| | (SEQ ID NO: 10) |
| Variant 8b | MRKLRRLKRKIAHKVKKYFKLSQRSIDRLVGVHPDLVKVVHRALELTPV |
| | DFGITEGVRSLETQKKYVAEGKSKTMKSRHLHGLAVDVVAYPKDKDTW |
| | NMKYYRMIADAFKQAGRELGVSVEWGGDWVSFKDGVHFQLPHSKYP |
| | (SEQ ID NO: 11) |
| Variant 8c | MRKLRRLKRKIAHKVKKYFKLSQRSYDRLVGVHPDLVKVVHRALELTP |
| | VDFGITEGVRSLETQKKYVAEGKSKTMKSRHLHGLAVDVVAYPKDKDT |
| | WNMKYYRMIADAFKQAGRELGVSVEWGGDWVSFKDGVHFQLPHSKYP |
| | (SEQ ID NO: 12) |
| Variant 8d | MRKLRRLKRKIAHKVKKYFKLSQRSADRLVGVHPDLVKVVHRALELTP |
| | VDFGITEGVRSLETQKKYVAEGKSKTMKSRHLHGLAVDVVAYPKDKDT |
| | WNMKYYRMIADAFKQAGRELGVSVEWGGDWVSFKDGVHFQLPHSKYP |
| | (SEQ ID NO: 13) |
| Variant 8e | MRKLRRLKRKIAHKVKKYFKLSQRSPDRLVGVHPDLVKVVHRALELTPV |
| | DFGITEGVRSLETQKKYVAEGKSKTMKSRHLHGLAVDVVAYPKDKDTW |
| | NMKYYRMIADAFKQAGRELGVSVEWGGDWVSFKDGVHFQLPHSKYP |
| | (SEQ ID NO: 14) |
| Variant 8f | MRKLRRLKRKIAHKVKKYFKLSQRSRDRLVGVHPDLVKVVHRALELTP |
| | VDFGITEGVRSLETQKKYVAEGKSKTMKSRHLHGLAVDVVAYPKDKDT |
| | WNMKYYRMIADAFKQAGRELGVSVEWGGDWVSFKDGVHFQLPHSKYP |
| | (SEQ ID NO: 15) |
| Variant 8g | MRKLRRLKRKIAHKVKKYFKLSQRSDDRLVGVHPDLVKVVHRALELTP |
| | VDFGITEGVRSLETQKKYVAEGKSKTMKSRHLHGLAVDVVAYPKDKDT |
| | WNMKYYRMIADAFKQAGRELGVSVEWGGDWVSFKDGVHFQLPHSKYP |
| | (SEQ ID NO: 16) |
| Variant 8h | MRKLRRLKRKIAHKVKKYFKLSQRSSDRLVGVHPDLVKVVHRALELTPV |
| | DFGITEGVRSLETQKKYVAEGKSKTMKSRHLHGLAVDVVAYPKDKDTW |
| | NMKYYRMIADAFKQAGRELGVSVEWGGDWVSFKDGVHFQLPHSKYP |
| | (SEQ ID NO: 17) |
| Variant 8i | MRKLRRLKRKIAHKVKKYFKLSQRSNDRLVGVHPDLVKVVHRALELTP |
| | VDFGITEGVRSLETQKKYVAEGKSKTMKSRHLHGLAVDVVAYPKDKDT |
| | WNMKYYRMIADAFKQAGRELGVSVEWGGDWVSFKDGVHFQLPHSKYP |
| | (SEQ ID NO: 18) |

In some embodiments, the LysECD7 variant is one that has a substitution at position 33 (H) of the wild-type sequence wherein the substitution is selected from the following group of amino acids: A, R, N, D, C, Q, E, G, I, L, K, M, F, P, S, T, W, Y, and V. Such sequences are shown below in Table 4.

TABLE 4

| | Sequence |
|---|---|
| Variant 9a | MRKLRRLKRKIAHKVKKYFKLSQRSKDRLVGVAPDLVKVVHRALELTP |
| | VDFGITEGVRSLETQKKYVAEGKSKTMKSRHLHGLAVDVVAYPKDKDT |
| | WNMKYYRMIADAFKQAGRELGVSVEWGGDWVSFKDGVHFQLPHSKYP |
| | (SEQ ID NO: 19) |
| Variant 9b | MRKLRRLKRKIAHKVKKYFKLSQRSKDRLVGVRPDLVKVVHRALELTP |
| | VDFGITEGVRSLETQKKYVAEGKSKTMKSRHLHGLAVDVVAYPKDKDT |
| | WNMKYYRMIADAFKQAGRELGVSVEWGGDWVSFKDGVHFQLPHSKYP |
| | (SEQ ID NO: 20) |
| Variant 9c | MRKLRRLKRKIAHKVKKYFKLSQRSKDRLVGVNPDLVKVVHRALELTP |
| | VDFGITEGVRSLETQKKYVAEGKSKTMKSRHLHGLAVDVVAYPKDKDT |
| | WNMKYYRMIADAFKQAGRELGVSVEWGGDWVSFKDGVHFQLPHSKYP |
| | (SEQ ID NO: 21) |
| Variant 9d | MRKLRRLKRKIAHKVKKYFKLSQRSKDRLVGVDPDLVKVVHRALELTP |
| | VDFGITEGVRSLETQKKYVAEGKSKTMKSRHLHGLAVDVVAYPKDKDT |
| | WNMKYYRMIADAFKQAGRELGVSVEWGGDWVSFKDGVHFQLPHSKYP |
| | (SEQ ID NO: 22) |
| Variant 9e | MRKLRRLKRKIAHKVKKYFKLSQRSKDRLVGVCPDLVKVVHRALELTP |
| | VDFGITEGVRSLETQKKYVAEGKSKTMKSRHLHGLAVDVVAYPKDKDT |
| | WNMKYYRMIADAFKQAGRELGVSVEWGGDWVSFKDGVHFQLPHSKYP |
| | (SEQ ID NO: 23) |
| Variant 9f | MRKLRRLKRKIAHKVKKYFKLSQRSKDRLVGVQPDLVKVVHRALELTP |
| | VDFGITEGVRSLETQKKYVAEGKSKTMKSRHLHGLAVDVVAYPKDKDT |
| | WNMKYYRMIADAFKQAGRELGVSVEWGGDWVSFKDGVHFQLPHSKYP |
| | (SEQ ID NO: 24) |
| Variant 9g | MRKLRRLKRKIAHKVKKYFKLSQRSKDRLVGVEPDLVKVVHRALELTP |
| | VDFGITEGVRSLETQKKYVAEGKSKTMKSRHLHGLAVDVVAYPKDKDT |
| | WNMKYYRMIADAFKQAGRELGVSVEWGGDWVSFKDGVHFQLPHSKYP |
| | (SEQ ID NO: 25) |
| Variant 9h | MRKLRRLKRKIAHKVKKYFKLSQRSKDRLVGVGPDLVKVVHRALELTP |
| | VDFGITEGVRSLETQKKYVAEGKSKTMKSRHLHGLAVDVVAYPKDKDT |
| | WNMKYYRMIADAFKQAGRELGVSVEWGGDWVSFKDGVHFQLPHSKYP |
| | (SEQ ID NO: 26) |
| Variant 9i | MRKLRRLKRKIAHKVKKYFKLSQRSKDRLVGVIPDLVKVVHRALELTPV |
| | DFGITEGVRSLETQKKYVAEGKSKTMKSRHLHGLAVDVVAYPKDKDTW |
| | NMKYYRMIADAFKQAGRELGVSVEWGGDWVSFKDGVHFQLPHSKYP |
| | (SEQ ID NO: 27) |
| Variant 9j | MRKLRRLKRKIAHKVKKYFKLSQRSKDRLVGVLPDLVKVVHRALELTP |
| | VDFGITEGVRSLETQKKYVAEGKSKTMKSRHLHGLAVDVVAYPKDKDT |
| | WNMKYYRMIADAFKQAGRELGVSVEWGGDWVSFKDGVHFQLPHSKYP |
| | (SEQ ID NO: 28) |
| Variant 9k | MRKLRRLKRKIAHKVKKYFKLSQRSKDRLVGVKPDLVKVVHRALELTP |
| | VDFGITEGVRSLETQKKYVAEGKSKTMKSRHLHGLAVDVVAYPKDKDT |
| | WNMKYYRMIADAFKQAGRELGVSVEWGGDWVSFKDGVHFQLPHSKYP |
| | (SEQ ID NO: 29) |
| Variant 9l | MRKLRRLKRKIAHKVKKYFKLSQRSKDRLVGVMPDLVKVVHRALELTP |
| | VDFGITEGVRSLETQKKYVAEGKSKTMKSRHLHGLAVDVVAYPKDKDT |
| | WNMKYYRMIADAFKQAGRELGVSVEWGGDWVSFKDGVHFQLPHSKYP |
| | (SEQ ID NO: 30) |
| Variant 9m | MRKLRRLKRKIAHKVKKYFKLSQRSKDRLVGVFPDLVKVVHRALELTPV |
| | DFGITEGVRSLETQKKYVAEGKSKTMKSRHLHGLAVDVVAYPKDKDTW |
| | NMKYYRMIADAFKQAGRELGVSVEWGGDWVSFKDGVHFQLPHSKYP |
| | (SEQ ID NO: 31) |
| Variant 9n | MRKLRRLKRKIAHKVKKYFKLSQRSKDRLVGVPPDLVKVVHRALELTPV |
| | DFGITEGVRSLETQKKYVAEGKSKTMKSRHLHGLAVDVVAYPKDKDTW |
| | NMKYYRMIADAFKQAGRELGVSVEWGGDWVSFKDGVHFQLPHSKYP |
| | (SEQ ID NO: 32) |
| Variant 9o | MRKLRRLKRKIAHKVKKYFKLSQRSKDRLVGVSPDLVKVVHRALELTPV |
| | DFGITEGVRSLETQKKYVAEGKSKTMKSRHLHGLAVDVVAYPKDKDTW |
| | NMKYYRMIADAFKQAGRELGVSVEWGGDWVSFKDGVHFQLPHSKYP |
| | (SEQ ID NO: 33) |
| Variant 9p | MRKLRRLKRKIAHKVKKYFKLSQRSKDRLVGVTPDLVKVVHRALELTP |
| | VDFGITEGVRSLETQKKYVAEGKSKTMKSRHLHGLAVDVVAYPKDKDT |
| | WNMKYYRMIADAFKQAGRELGVSVEWGGDWVSFKDGVHFQLPHSKYP |
| | (SEQ ID NO: 34) |
| Variant 9q | MRKLRRLKRKIAHKVKKYFKLSQRSKDRLVGVWPDLVKVVHRALELTP |
| | VDFGITEGVRSLETQKKYVAEGKSKTMKSRHLHGLAVDVVAYPKDKDT |
| | WNMKYYRMIADAFKQAGRELGVSVEWGGDWVSFKDGVHFQLPHSKYP |
| | (SEQ ID NO: 35) |
| Variant 9r | MRKLRRLKRKIAHKVKKYFKLSQRSKDRLVGVYPDLVKVVHRALELTP |
| | VDFGITEGVRSLETQKKYVAEGKSKTMKSRHLHGLAVDVVAYPKDKDT |
| | WNMKYYRMIADAFKQAGRELGVSVEWGGDWVSFKDGVHFQLPHSKYP |
| | (SEQ ID NO: 36) |
| Variant 9s | MRKLRRLKRKIAHKVKKYFKLSQRSKDRLVGVVPDLVKVVHRALELTP |
| | VDFGITEGVRSLETQKKYVAEGKSKTMKSRHLHGLAVDVVAYPKDKDT |
| | WNMKYYRMIADAFKQAGRELGVSVEWGGDWVSFKDGVHFQLPHSKYP |
| | (SEQ ID NO: 37) |

In some embodiments, the LysECD7 variant is one that has a substitution at position 120 (V) of the wild-type sequence wherein the substitution is selected from the following group of amino acids: G, A, L, I, P, F, M, W, R, N, D, C, Q, E, H, K, S, T, and Y. Such sequences are shown below in Table 5.

TABLE 5

| | Sequence |
|---|---|
| Variant 10a | MRKLRRLKRKIAHKVKKYFKLSQRSKDRLVGVHPDLVKVVHRALELT |
| | PVDFGITEGVRSLETQKKYVAEGKSKTMKSRHLHGLAVDVVAYPKDK |
| | DTWNMKYYRMIADAFKQAGRELGVSGEWGGDWVSFKDGVHFQLPHS |
| | KY (SEQ ID NO: 38) |
| Variant 10b | MRKLRRLKRKIAHKVKKYFKLSQRSKDRLVGVHPDLVKVVHRALELT |
| | PVDFGITEGVRSLETQKKYVAEGKSKTMKSRHLHGLAVDVVAYPKDK |
| | DTWNMKYYRMIADAFKQAGRELGVSAEWGGDWVSFKDGVHFQLPHS |
| | KY (SEQ ID NO: 39) |
| Variant 10c | MRKLRRLKRKIAHKVKKYFKLSQRSKDRLVGVHPDLVKVVHRALELT |
| | PVDFGITEGVRSLETQKKYVAEGKSKTMKSRHLHGLAVDVVAYPKDK |
| | DTWNMKYYRMIADAFKQAGRELGVSLEWGGDWVSFKDGVHFQLPHS |
| | KY (SEQ ID NO: 40) |
| Variant 10d | MRKLRRLKRKIAHKVKKYFKLSQRSKDRLVGVHPDLVKVVHRALELT |
| | PVDFGITEGVRSLETQKKYVAEGKSKTMKSRHLHGLAVDVVAYPKDK |
| | DTWNMKYYRMIADAFKQAGRELGVSIEWGGDWVSFKDGVHFQLPHS |
| | KY (SEQ ID NO: 41) |
| Variant 10e | MRKLRRLKRKIAHKVKKYFKLSQRSKDRLVGVHPDLVKVVHRALELT |
| | PVDFGITEGVRSLETQKKYVAEGKSKTMKSRHLHGLAVDVVAYPKDK |
| | DTWNMKYYRMIADAFKQAGRELGVSPEWGGDWVSFKDGVHFQLPHS |
| | KY (SEQ ID NO: 42) |
| Variant 10f | MRKLRRLKRKIAHKVKKYFKLSQRSKDRLVGVHPDLVKVVHRALELT |
| | PVDFGITEGVRSLETQKKYVAEGKSKTMKSRHLHGLAVDVVAYPKDK |
| | DTWNMKYYRMIADAFKQAGRELGVSFEWGGDWVSFKDGVHFQLPHS |
| | KY (SEQ ID NO: 43) |
| Variant 10g | MRKLRRLKRKIAHKVKKYFKLSQRSKDRLVGVHPDLVKVVHRALELT |
| | PVDFGITEGVRSLETQKKYVAEGKSKTMKSRHLHGLAVDVVAYPKDK |
| | DTWNMKYYRMIADAFKQAGRELGVSMEWGGDWVSFKDGVHFQLPH |
| | SKY (SEQ ID NO: 44) |
| Variant 10h | MRKLRRLKRKIAHKVKKYFKLSQRSKDRLVGVHPDLVKVVHRALELT |
| | PVDFGITEGVRSLETQKKYVAEGKSKTMKSRHLHGLAVDVVAYPKDK |
| | DTWNMKYYRMIADAFKQAGRELGVSWEWGGDWVSFKDGVHFQLPH |
| | SKY (SEQ ID NO: 45) |
| Variant 10i | MRKLRRLKRKIAHKVKKYFKLSQRSKDRLVGVHPDLVKVVHRALELT |
| | PVDFGITEGVRSLETQKKYVAEGKSKTMKSRHLHGLAVDVVAYPKDK |
| | DTWNMKYYRMIADAFKQAGRELGVSREWGGDWVSFKDGVHFQLPHS |
| | KY (SEQ ID NO: 46) |
| Variant 10j | MRKLRRLKRKIAHKVKKYFKLSQRSKDRLVGVHPDLVKVVHRALELT |
| | PVDFGITEGVRSLETQKKYVAEGKSKTMKSRHLHGLAVDVVAYPKDK |
| | DTWNMKYYRMIADAFKQAGRELGVSNEWGGDWVSFKDGVHFQLPHS |
| | KY (SEQ ID NO: 47) |
| Variant 10k | MRKLRRLKRKIAHKVKKYFKLSQRSKDRLVGVHPDLVKVVHRALELT |
| | PVDFGITEGVRSLETQKKYVAEGKSKTMKSRHLHGLAVDVVAYPKDK |
| | DTWNMKYYRMIADAFKQAGRELGVSDEWGGDWVSFKDGVHFQLPHS |
| | KY (SEQ ID NO: 48) |
| Variant 10l | MRKLRRLKRKIAHKVKKYFKLSQRSKDRLVGVHPDLVKVVHRALELT |
| | PVDFGITEGVRSLETQKKYVAEGKSKTMKSRHLHGLAVDVVAYPKDK |
| | DTWNMKYYRMIADAFKQAGRELGVSCEWGGDWVSFKDGVHFQLPHS |
| | KY (SEQ ID NO: 49) |
| Variant 10m | MRKLRRLKRKIAHKVKKYFKLSQRSKDRLVGVHPDLVKVVHRALELT |
| | PVDFGITEGVRSLETQKKYVAEGKSKTMKSRHLHGLAVDVVAYPKDK |
| | DTWNMKYYRMIADAFKQAGRELGVSQEWGGDWVSFKDGVHFQLPHS |
| | KY (SEQ ID NO: 50) |
| Variant 10n | MRKLRRLKRKIAHKVKKYFKLSQRSKDRLVGVHPDLVKVVHRALELT |
| | PVDFGITEGVRSLETQKKYVAEGKSKTMKSRHLHGLAVDVVAYPKDK |
| | DTWNMKYYRMIADAFKQAGRELGVSEEWGGDWVSFKDGVHFQLPHS |
| | KY (SEQ ID NO: 51) |
| Variant 10o | MRKLRRLKRKIAHKVKKYFKLSQRSKDRLVGVHPDLVKVVHRALELT |
| | PVDFGITEGVRSLETQKKYVAEGKSKTMKSRHLHGLAVDVVAYPKDK |
| | DTWNMKYYRMIADAFKQAGRELGVSHEWGGDWVSFKDGVHFQLPHS |
| | KY (SEQ ID NO: 52) |
| Variant 10p | MRKLRRLKRKIAHKVKKYFKLSQRSKDRLVGVHPDLVKVVHRALELT |
| | PVDFGITEGVRSLETQKKYVAEGKSKTMKSRHLHGLAVDVVAYPKDK |
| | DTWNMKYYRMIADAFKQAGRELGVSKEWGGDWVSFKDGVHFQLPHS |
| | KY (SEQ ID NO: 53) |
| Variant 10q | MRKLRRLKRKIAHKVKKYFKLSQRSKDRLVGVHPDLVKVVHRALELT |
| | PVDFGITEGVRSLETQKKYVAEGKSKTMKSRHLHGLAVDVVAYPKDK |
| | DTWNMKYYRMIADAFKQAGRELGVSSEWGGDWVSFKDGVHFQLPHS |
| | KY (SEQ ID NO: 54) |
| Variant 10r | MRKLRRLKRKIAHKVKKYFKLSQRSKDRLVGVHPDLVKVVHRALELT |
| | PVDFGITEGVRSLETQKKYVAEGKSKTMKSRHLHGLAVDVVAYPKDK |
| | DTWNMKYYRMIADAFKQAGRELGVSTEWGGDWVSFKDGVHFQLPHS |
| | KY (SEQ ID NO: 55) |
| Variant 10s | MRKLRRLKRKIAHKVKKYFKLSQRSKDRLVGVHPDLVKVVHRALELT |
| | PVDFGITEGVRSLETQKKYVAEGKSKTMKSRHLHGLAVDVVAYPKDK |
| | DTWNMKYYRMIADAFKQAGRELGVSYEWGGDWVSFKDGVHFQLPHS |
| | KY (SEQ ID NO: 56) |

Other embodiments comprise fragments of the above-described LysECD7 variants. A LysECD7 variant fragment comprises or consists of a portion of the peptide according to any one of SEQ ID Nos: 2 to 7 and 9 to 56, wherein the portion comprises or consists of residues one or more regions selected from 25-40, 68-73, 25-73, 25-110, 25-131, 68-110, 68-131, 97-110, 97-131, or 119-131, wherein the fragment comprises at least one mutation.

Other embodiments include polynucleotides that encode the LysECD7 variants described herein or the LysECD7 variant fragment described herein. Due to the degeneracy of nucleotide coding sequences, multiple DNA sequences can encode the same amino acid sequence and are also contemplated.

Another embodiment is a polynucleotide that comprises a sequence that hybridizes with a polynucleotide that encodes LysECD7 variant fragment as described above. These hybridizable polynucleotides can comprise 8 to 50 or 10 to 24 nucleotides. Such hybridizable polynucleotides can be used in nucleic acid hybridization-based assays such as PCR, isothermal amplification, and the like.

Another embodiment is fusion protein comprising one or more anti-microbial peptides selected from a wt LysECD7 peptide, wt LysECD7-SMAP29 fusion peptide, or LysECD7 variant peptide as described herein (which includes SMAP fusions) and a spore crust protein selected from CotV, CotW, CotX, CotY, CotZ, and CgeA of B. subtilis, wherein the one or more peptides are N-terminally attached, C-terminally attached, or both N-terminally and C-terminally attached to the spore crust protein. In an embodiment, the spore crust protein is CotY. In another embodiment, the spore crust protein is CotX. In another embodiment, the spore crust protein is CotZ. In another embodiment, the spore crust protein is CotW. In another embodiment, the spore crust protein is CotV. In an embodiment, two or three anti-microbial peptides are attached to the spore crust protein at its N-terminal end.

Another embodiment of the present disclosure is a vector comprising for expression a polynucleotide or a polynucleotide encoding the LysECD7 variants described herein or encoding the fusion protein described above. In an embodiment, the polynucleotide encodes a peptide having an amino acid sequence selected from SEQ ID Nos: 2 to 7 and 9 to 56. In an embodiment, the polynucleotide encodes a peptide having an amino acid sequence selected from SEQ ID Nos: 2 to 7 and 9 to 56 and a SMAP29 peptide (SEQ ID NO: 8). In an embodiment, the vector further comprises for expression a promoter, such as a promoter selected from P_{ylb}, P_{cotYZ}, P_{cotVWX}, and P_{cotX}. The sequences of the described promoters are known to those in the field. The following are sequences for promoters Pylb, PcotYZ, and PcotVWX, wherein the start codon is the last three nucleotides in bold, italic font.

TABLE 6

| Promoter | Sequence |
|---|---|
| PcotVWX | ATGAATTGTATCTTGTCCGGCAGAAAATTGGTTATTTTTA |
| | TTATTCCGCTCTGCACCCCATTTGCATTATATAGAGTATG |
| | GATAAGAAAGGAGTGAAGGTTATG (SEQ ID NO: 57) |
| PcotYZ | ACGCTCTGCTTTTTCTTATTTTCCAAGCATATGATGAATA |
| | TATAGACGTTCACCCACACCAAGTGGGGCACGGGTACATA |
| | TGTTGTTAAGGACTAAAGTCAAATACCCTATAATTCTAGA |
| | AAGGAGGTGGCCGGCATG (SEQ ID NO: 58) |
| Pylb | AAGTTTAAATATTTGGATTTTTTAAATAAAGCGTTTACAA |
| | TATATGTAGAAACAACAAAGGGGGAGATTTGTATG |
| | (SEQ ID NO: 59) |

Optionally, a linker is located between the anti-microbial peptide and the spore crust protein. The linker can be a peptide sequence of between 10 and 20 amino acid residues to create a physical distance between the anti-microbial peptide and the spore crust protein to mitigate interference with the activity by the fusion partner. The linker can be rigid or flexible. In an embodiment, the linker has a secondary structure that is an alpha-helical structure, such as a linker having a sequence GGGEAAAKGGG (SEQ ID NO: 60).

Another embodiment of the present disclosure is a method of detecting a polynucleotide described herein, comprising contacting the polynucleotide with a reference sequence that is configured to hybridize with at least portion of the polynucleotide, wherein the portion hybridizes with the portion of the sequence that encodes amino acid residues 25-40, 68-73, 97-110, 119-131, or a combination thereof of the encoded LysECD7 variant peptide. Another embodiment of the present disclosure is a method of detecting a LysECD7 variant described herein, comprising contacting the variant with a reference sequence that is configured to hybridize with at least portion of the variant, wherein the reference sequence has a sequence that hybridizes with the amino acid residues 25-40, 68-73, 97-110, 119-131, or a combination thereof of the LysECD7 variant.

Another embodiment of the present disclosure is a spore having an anti-microbial peptide selected from a wt LysECD7 peptide, wt LysECD7-SMAP29 fusion peptide, or LysECD7 variant peptide as described herein conjugated to the surface of the spore or wherein the spore is modified to express the anti-microbial peptide on the surface of the spore. In an embodiment, the spore comprises the expression cassette of the vector as described herein. In an embodiment, the genome of the spore is modified with the vector as described herein. In an embodiment, the spore is a spore according to PCT Publication WO2020243730A1, which is hereby incorporated by reference in its entirety.

In an embodiment, the spore further comprises one or more recombinant biological barcodes that can be utilized to track and identify the spore. The spore can be mixed or applied to an object that moves through a supply chain and then subsequently extracted from the object on which it is mixed or applied to determine the barcode. The biological barcode can consist of 12-1000 nucleotides. In an embodiment, at least one of the one or more recombinant biological barcodes is a nucleic acid sequence comprising one or more barcode regions, wherein the barcode region consists of a series of N nucleotides that are not present in the wild-type spore or any other region of the biological barcode and differs by three or more nucleotides from a series of N nucleotides in the wild-type spore or any other region of the biological barcode, wherein N is at least 12.

In an embodiment, the biological barcode comprises a barcode region or comprises one or more conserved regions and one or more barcode regions. The conserved region is useful so that a single primer can be used to detect more than one biological barcode. For example, in a system embodiment, a first object has applied thereto a first spore as described herein and a second object has applied thereto a second spore, wherein each comprises its own unique biological barcode. In an embodiment wherein the biological barcode comprises a conserved region and a barcode region, the conserved regions have an identical sequence such that a single primer can be used to hybridize with both the first and second biological barcodes for the purpose sequencing the barcode regions of the first spore and the second spore. The barcode region sequence obtained through sequencing can then be matched with a barcode sequence stored in a database of a plurality of barcode sequences, wherein the barcode sequence is associated with one of the first or second object. By way of matching the sequenced barcode region with the barcode sequence stored in the database, the identity of the first or second object can be confirmed.

In an embodiment, spores as described above can have a genome modified to render inoperable one or more genes that are needed for spore germination. The genome modified to render inoperable one or more genes that are needed for production of an essential metabolite or germination. The spores can be of a genus selected from Bacillus, Clostridium, and Saccharmoyces. The spores can be of a species selected from Bacillus subtilis, Bacillus cereus, Bacillus thuringiensis, Clostridium difficile, Clostridium perfringens, and Saccharomyces cerevisiae.

In an embodiment, the species is B. subtilis, and the genome does not express at least one of or any combination of sleB, cwlD, and cwlJ. In an embodiment, the genome does not express gerD. In an embodiment, the genome does not express a gene selected from the gerA operon, gerAA, gerAB, gerB operon, gerC, gerK operon, gerP, gerT, gerM, gerQ, gerE, ypeB, pdaA, cotH, cotG, cotB, cotE, cotT, spoVAC, spoVAD, spoVAE, and sscA. In an embodiment, the genome does not express a gene encoding a germinant nutrient receptor and/or a cell wall lytic enzyme.

Another embodiment is a method of inhibiting microbial growth comprising applying a growth-inhibiting amount of a LysECD7 variant as described herein or a spore containing an anti-microbial peptide to an object. In an embodiment, the object is edible. In an embodiment, the object is a crop. In an embodiment, the object is a food crop, such as an apple or a leafy green like lettuce or spinach. In another embodiment, the object is a processed food such as ice cream or olive oil. In an embodiment, the application of the peptide comprises spraying the formulation described below to the object. The microbial growth that is inhibited can be the growth of Gram negative bacteria and optionally, a Gram positive bacteria by the activity of the SMAP29 peptide or the growth of at least E. coli and/or Salmonella. In an embodiment, the LysECD7 variant is part of a spore. The spore as described herein can applied to the object, wherein the anti-microbial peptide, such as a LysECD7 variant, is conjugated to or expressed on the surface of the spore. This method can reduce the likelihood of food-borne illness associated with microbial growth when the object is an edible object.

Another embodiment is a method of treating a subject for a microbial infection, reducing the incidence of a microbial infection (e.g., preventing) comprising administering to a subject in need thereof a therapeutic amount of a LysECD7 variant to the subject. In an embodiment, the subject is a human, livestock, a pet, or a mammalian pet.

Another embodiment is formulation comprising an LysECD7 variant as described herein or a spore as described herein. In an embodiment, the formulation is an aqueous solution that is suitable for spraying, such as on a crop. In an embodiment, the formulation comprises or consists of water.

### EXAMPLES

### Example 1

### Mutation Site Determination:

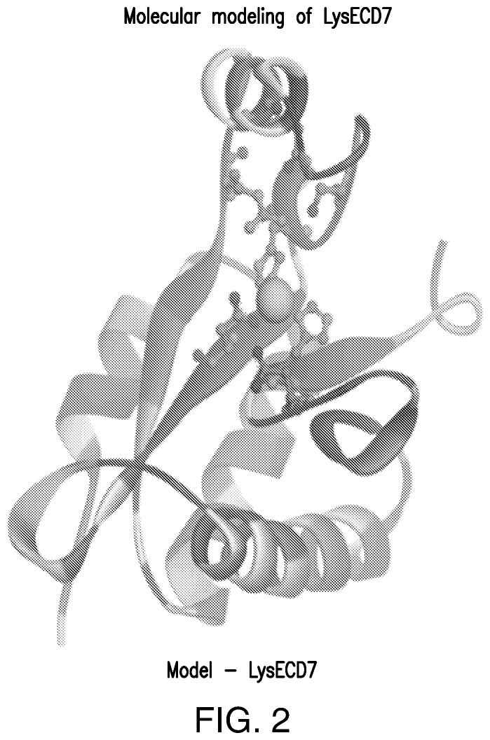

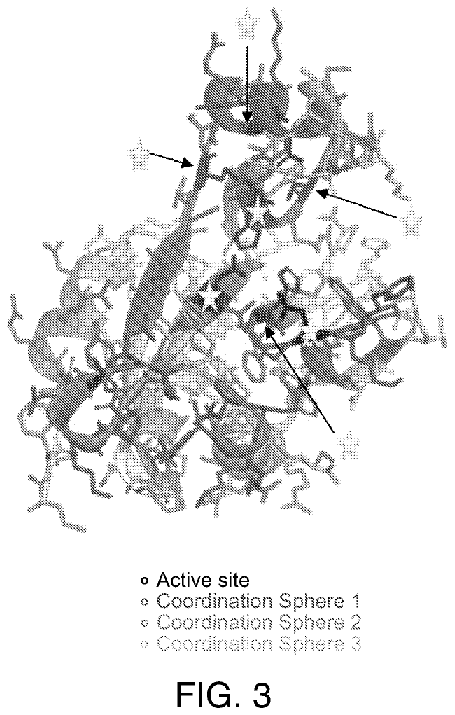

The following protocol was used to determine the regions of LysECD7 where amino acid changes would be most likely to result in altered function. First, the nucleic acid sequence of wild type LysECD7 was compared to all known protein sequences using the BLAST tool (publicly available at ncbi.nlm.nih.gov). Candidate proteins showing significant amino acid homology with LysECD7 that also had crystal structure data available were identified, and the Serratia marcescens L-Ala D-Glu endopeptidase ChiXa (5OPD) was chosen as the closest match. Amino acid positions to be varied were determined by creating a three dimensional model of the LysECD7 protein based on the crystal structure of ChiXa whose amino acid sequence is 49% homologous with LysECD7 (FIG. 2). Five separate mutation zones were identified and targeted for mutation (FIG. 3) and these zones are marked by arrows in the amino acid sequence shown in FIG. 1. Fifty five amino acid positions were mutated for a total of 1100 variants. LysECD7 variants with an optimized SMAP29 region were screened in pools for altered antimicrobial activity and critical amino acids identified to create a suite of variants with titrated activities.

LysECD7 Variants with Increased or Decreased Lysis Activity Against E. coli:

Five mutation zones within the LysECD7 protein were identified as shown in FIG. 1. For each amino acid position within the mutation zones, DNA encoding variants of LysECD7 with an optimized SMAP region as depicted in FIG. 1 were chemically synthesized in pools with the modifications at the X positions in SEQ ID NO:9. Each pool contained an identical set of encoded LysECD7 variants, except that one amino acid position was modified to have a different amino acid and all 20 amino acids were represented in the pool. Codons were optimized for expression in E. coli. The expression construct comprised T7 promoter, E. coli ribosome binding site, and transcriptional terminator so that the DNA could be transcribed and translated using a cell-free system.

The pools were used to create double stranded DNA that was tethered to Streptavidin Coated Polystyrene Particles (Spherotech #SVP 150-4) in a manner consistent with the manufacturer's instructions. Briefly, polymerase chain reaction (PCR) amplification was used to amplify the DNA with a biotin label added to the 5′ end of the reverse primer and the amplified DNA attached to the Streptavidin Coated Polystyrene Particles (SCPPs) in the following manner. The desired amount of SCPPs is washed 3× in an equal volume of phosphate buffered saline containing 0.5% Tween 20 (PBST).

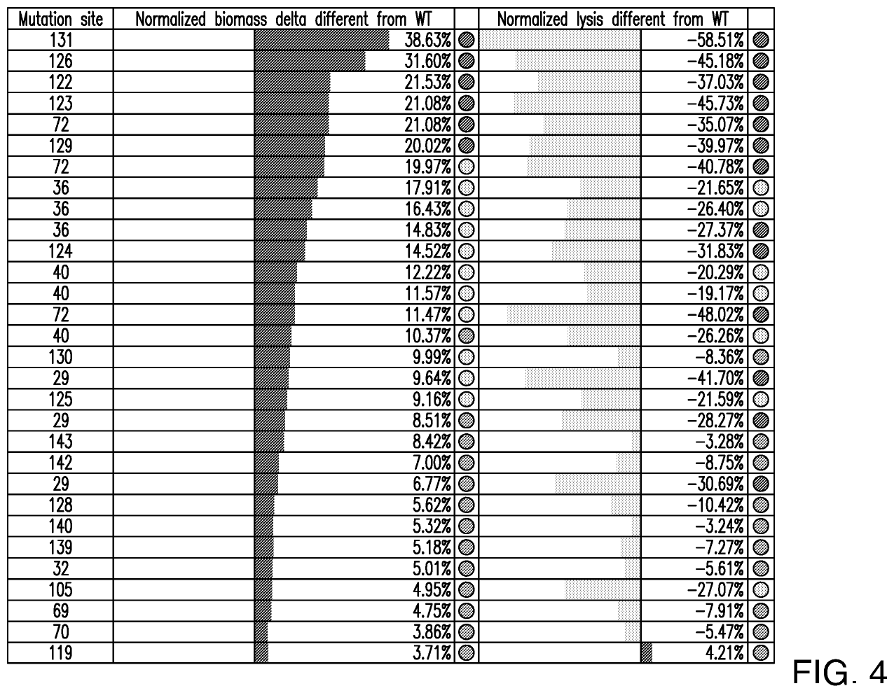

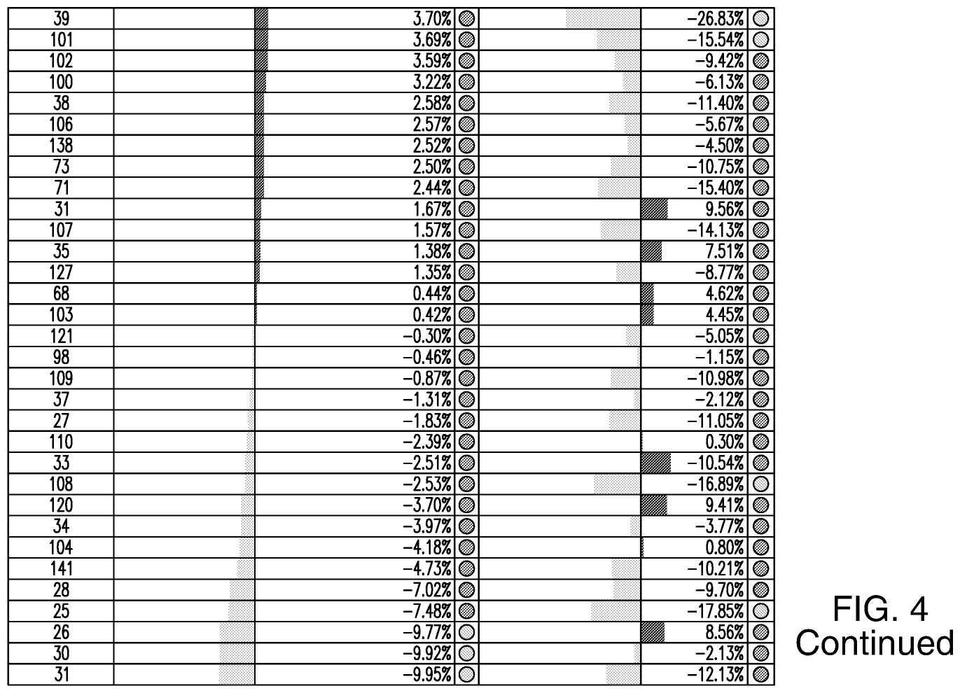

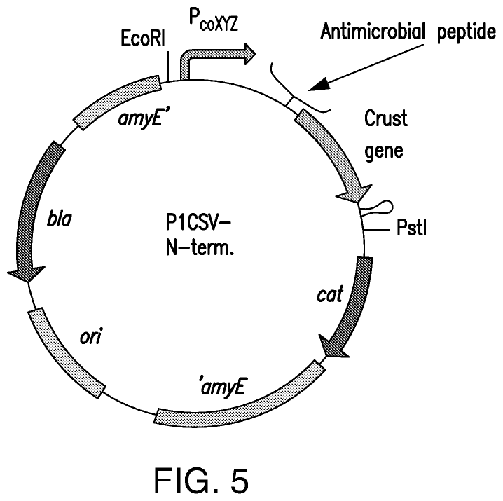

After final wash the SCPPs are re-suspended in PBST and biotinylated DNA added in excess, then incubated at room temperature with end-over-end rotation for 30 minutes. The SCPPs are then washed 3× with PBST and re-suspended at the desired concentration in PBST and stored at 4C. Successful attachment was verified using PCR primers that recognized portions of LysECD7 variants outside the mutation zones. The ‘wild type’ LysECD7 with optimized SMAP was also synthesized, tethered to SCPPs, and used as a control in the experiments. The NEBExpress® Cell-free E. coli Protein Synthesis System was used to transcribe and translate the LysECD7 variants which were then assayed for their ability to lyse E. coli. The E. coli used in the assay were engineered to express fluorescent protein and lysis was monitored both by disappearance of the intact bacterial cells and by release of fluorescent protein into the media. In the region comprising amino acids 25-37, several amino acid positions showed either a significant increase or decrease in lysis activity when compared to the ‘wild type’ LysECD7-SMAP as shown in FIG. 4. The table in FIG. 4 ranks the variants based on the p values represented by red dot (<0.01) yellow dot (<0.05) or gray dot (>0.05). The P-value was using unpaired t-test between each variant and the wild type tested at the same time for normalized biomass delta and normalized lysis.

### Example 3—Lysis Activity of Variants at Position 26

Variants with a substitution at position 26 were examined by cloning all 19 Lys-ECD7 mutants and the wild type sequence separately into a plasmid vector that adds a 6×His tag for purification and places the DNA sequence under control of T7 promoter that is IPTG inducible. The plasmids were used to transform E. coli and the proteins expressed. The insoluble fractions were collected and found to contain the Lys-ECD7 mutant proteins. After adjusting for the protein concentration the mutants were tested for their ability to lyse E. coli compared to that of the wild type enzyme similarly expressed. As summarized in Table 7 below, several mutants showed increased ability to lyse E. coli cells.

TABLE 7

| | | | Mutants with higher |
|---|---|---|---|
| | Single amino acid | | lytic activity than |
| | substitution of Lysine | Codon base | LysECD7_SMAP29 |
| | (K) 26 | substitution | (WT) |
| 1 | Lysine (K) > Isoleucine (I) | AAA > ATC | ↑ |
| 2 | Lysine (K) > Arginine (R) | AAA > CGT | ↑ |
| 3 | Lysine (K) > Histidine (H) | AAA > CAC | |
| 4 | Lysine (K) > Glutamine(Q) | AAA > CAG | |
| 5 | Lysine (K) > Aspartate(D) | AAA > GAC | ↑ |
| 6 | Lysine (K) > Alanine (A) | AAA > GCT | ↑ |
| 7 | Lysine (K) > Valine (V) | AAA > GTT | |
| 8 | Lysine (K) > Serine (S) | AAA > AGC | ↑ |
| 9 | Lysine (K) > Cysteine (C) | AAA > TGC | ↑ |
| 10 | Lysine (K) > Tyrosine (Y) | AAA > TAC | ↑ |
| 11 | Lysine (K) > Proline (P) | AAA > CCG | ↑ |
| 12 | Lysine (K) > Tryptophan | AAA > TGG | |
| | (W) | | |
| 13 | Lysine (K) > Leucine (L) | AAA > CTG | |
| 14 | Lysine (K) > Glycine (G) | AAA > GGT | |
| 15 | Lysine (K) > Asparagine | AAA > AAC | ↑ |
| | (N) | | |
| 16 | Lysine (K) > Glutamate (E) | AAA > GAA | |
| 17 | Lysine (K) > Phenylalanine | AAA > TTC | |
| | (F) | | |
| 18 | Lysine (K) > Threonine (T) | AAA > ACC | |
| 19 | Lysine (K) > Serine (S) | AAA > TCT | ↑ |

### Example 4—Spore Surface Display

This examples describes the development and expression of anti-bacterial chimeric proteins on the surface of a B. subtilis spore crust. Vectors were configured to express one of three chimeric peptides as follows: eGFP-LysECD7-SMAP29-GGGEAAAKGGG-CotY; eGFP-LysECD7-GGGEAAAKGGG-CotY; and LysECD7-SMAP29-CotY. For this experiment, all of the chimeric proteins comprise the CotY protein, as it is one of the most abundant proteins that make up the outer crust of the Bacillus subtilis spore. By expressing CotY as a fusion with LyseECD7 variants and optionally, SMAP29, the anti-microbial peptides are displayed on the spore crust.

The vector and methodology used to express the above-described fusion protein in B. subtilis is described in Bartels et al., Sporobeads: The Utilization of the Bacillus subtilis Endospore Crust as a Protein Display Platform. ACS Synth Biol. 2018 Feb. 16; 7 (2): 452-461, which is hereby incorporated by reference in its entirety. The vector system (p1CSV) described in Bartels et al. (2018) was used, in particular, the vector system wherein CotY is under the control of the PcotYZ promoter and the LysECD7 variant or LysECD7 plus SMAP29 fusion were located on the N-terminal side of CotY. As described in Bartels et al. (2018), the p1CSV vector is configured such that the expression cassette was integrated into the amyE locus of the B. subtilis genome. The sequence encoding the CotY protein is shown below.

| Spore Crust | |
|---|---|
| Protein | Sequence |
| CotY w/o | ATGAGCTGCGGAAAAACCCATGGCCGACATGAGAACTGTG |
| stop codon | TATGCGATGCAGTGGAAAAGATTTTAGCAGAGCAGGAGGC |
| | AGTTGAAGAACAGTGTCCGACTGGCTGCTATACCAACCTT |
| | TTAAACCCTACGATTGCTGGAAAAGACACAATTCCGTTTC |
| | TCGTTTTTGATAAAAAAGGCGGATTGTTCTCCACATTCGG |
| | AAACGTAGGGGGATTTGTGGATGATATGCAATGCTTTGAA |
| | TCCATTTTCTTCCGCGTCGAAAAATTATGCGATTGCTGTG |
| | CAACACTGTCTATTTTACGCCCGGTCGATGTCAAAGGCGA |
| | TACCTTAAGTGTTTGCCACCCTTGCGACCCGGATTTCTTC |
| | GGGCTAGAAAAAACAGATTTCTGCATTGAAGTGGATCTCG |
| | GATGCTTCTGCGCGATTCAGTGCCTGTCACCAGAGCTAGT |
| | TGACAGAACATCGCCTCACAAAGATAAAAAGCATCATCAC |
| | (SEQ ID NO: 61) |

Spores bearing the fusion proteins were made using DSM media according to a method published in Harwood C. M. and Cutting S. M., Molecular Biological Methods for Bacillus, Wiley 1990, which is hereby incorporated by reference in its entirety.

Expression of the LysECD7 fusion proteins were detected by Western blot using an anti-CotY antibody. Fusions were detected both with and without SMAP29. Spores comprising eGFP-LysECD7-SMAP29-CotY fusions showed fluorescence signal.

The spores generated demonstrated antibacterial activity in an initial assay.

### Example 5—Antibacterial Testing in Water

Purified LysECD7 variant according to SEQ ID NO: 11 (Variant 8b) showed antibacterial activity against three E. coli strains, namely, Turbo, BL21 (DE3), and Seattle 1946.

Dilutions of purified protein were mixed with between 4×10⁷ E. coli cells per ml in water, and after a 10 minute incubation period, dilutions of the mixture were spread on L-agar plates. The minimum inhibitory concentration of LysECD7 against these strains was 10 μg/ml against E. coli BL21 (DE3) and Seattle 1946.

### Example 6—Antibacterial Testing on the Surface of Lettuce

As described in this example, purified LysECD7 showed antibacterial activity against E. coli BL21 (DE3) on the surface of a leaf of Romaine lettuce. This was carried out by removing the natural microbiome of the lettuce by treatment with chlorinated water (200 mg/l Calcium hypochlorite). E. coli BL21 (DE3) cells were then applied to the surface by spotting. Purified LysECD7 was then spotted onto the surface of the leaf. Treated leaves were then digested with pectinase (90 U/ml) to release bacteria from the surface of the leaf, filtered, and then dilutions of the mixture were spread on L-agar plates. An aqueous solution of 250 μg/ml LysECD7 K26I (without SMAP) killed 70-80% of the E. coli BL21 (DE3) on the surface of the leaf compared to an untreated control. An aqueous solution of 25 μg/ml LysECD7 K26I (with SMAP) killed 82% of the E. coli BL21 (DE3) on the surface of the leaf compared to an untreated control.

### REFERENCES

1. “Antibiotic Resistance Threats in the United States 2019” Atlanta, GA: U.S. Department of Health and Human Services, CDC; 2019.
2. “No Time to Wait: Securing the Future From Drug-Resistant Infections”, Report to the Secretary General of the United Nations by the Interagency Coordination Group on Antimicrobial Resistance, April 2019.
3. Brown, E., Wright, G. Antibacterial drug discovery in the resistance era. Nature 529, 336-343 (2016). https://doi.org/10.1038/nature17042
4. Kumar, Prashant, Jayachandran N. Kizhakkedathu, and Suzana K. Straus. “Antimicrobial Peptides: Diversity, Mechanism of Action and Strategies to Improve the Activity and Biocompatibility In Vivo.” Biomolecules 8, no. 1 (March 2018): 4. https://doi.org/10.3390/biom8010004.
5. Antonova, Nataliia P., Daria V. Vasina, Anastasiya M. Lendel, Evgeny V. Usachev, Valentine V. Makarov, Alexander L. Gintsburg, Artem P. Tkachuk, and Vladimir A. Gushchin. “Broad Bactericidal Activity of the Myoviridae Bacteriophage Lysins LysAm24, LysECD7, and LysSi3 against Gram-Negative ESKAPE Pathogens.” Viruses 11, no. 3 (Mar. 21, 2019): E284. https://doi.org/10.3390/v11030284.
6. Vorob'ev, A. M., M. N. Anurova, A. V. Aleshkin, V. A. Gushchin, D. V. Vasina, N. P. Antonova, I. A. Kiseleva, et al. “Determination of Bactericidal Activity Spectrum of Recombinant Endolysins of ECD7, Am24, Ap22, Si3, and St11 Bacteriophages.” Bulletin of Experimental Biology and Medicine 170, no. 5 (March 2021): 636-39. https://doi.org/10.1007/s10517-021-05122-6.
7. Xu, Qian, Xuefeng Hu, and Yunbing Wang. “Alternatives to Conventional Antibiotic Therapy: Potential Therapeutic Strategies of Combating Antimicrobial-Resistance and Biofilm-Related Infections.” Molecular Biotechnology, Jul. 26, 2021. https://doi.org/10.1007/s12033-021-00371-2.
8. Antonova, Nataliia P., Daria V. Vasina, Evgeny O. Rubalsky, Mikhail V. Fursov, Alina S. Savinova, Igor V. Grigoriev, Evgeny V. Usachev, et al. “Modulation of Endolysin LysECD7 Bactericidal Activity by Different Peptide Tag Fusion.” Biomolecules 10, no. 3 (Mar. 12, 2020): E440. https://doi.org/10.3390/biom10030440.
9. Shwaiki, Laila N., Aylin W. Sahin, and Elke K. Arendt. “Study on the Inhibitory Activity of a Synthetic Defensin Derived from Barley Endosperm against Common Food Spoilage Yeast.” Molecules (Basel, Switzerland) 26, no. 1 (Dec. 31, 2020): E165. https://doi.org/10.3390/molecules26010165.
10. Kraszewska, Joanna, Michael C. Beckett, Tharappel C. James, and Ursula Bond. “Comparative Analysis of the Antimicrobial Activities of Plant Defensin-Like and Ultrashort Peptides against Food-Spoiling Bacteria.” Applied and Environmental Microbiology 82, no. 14 (Jul. 15, 2016): 4288-98. https://doi.org/10.1128/AEM.00558-16.

1. A peptide comprising a sequence that is a LysECD7 variant of a LysECD7 wild-type sequence, wherein the wild type has a sequence as follows:
(SEQ ID NO: 1)
MRKLRRLKRKIAHKVKKYFKLSQRSKDRLVGVHPDLVKVVHRALELTPVDF
GITEGVRSLETQKKYVAEGKSKTMKSRHLHGLAVDVVAYPKDKDTWNMKYY
RMIADAFKQAGRELGVSVEWGGDWVSFKDGVHFQLPHSKYP
wherein the LysECD7 variant comprises a sequence SEQ ID NO: 1 with at least one amino acid residue selected from positions 25-40, 68-73, 97-110, 119-131, and 138-143 of SEQ ID NO: 1 substituted with a different amino acid.

2. The peptide according to claim 1 comprising a sequence according to the following:
(SEQ ID NO: 2) 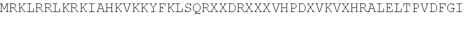 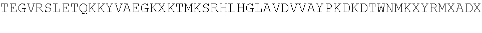 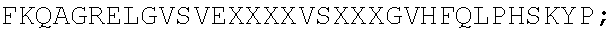
wherein X is independently selected from the following amino acids: A, R, N, D, C, Q, E, G, H, I, L, K, M, F, P, S, T, W, Y, and V and wherein at least one X is not the same amino acid as that in the same position in SEQ ID NO: 1.

3. The peptide according to claim 1 comprising a sequence according to the following:
(SEQ ID NO: 3)
MRKLRRLKRKIAHKVKKYFKLSQRSKDRLVGVHPDLVKVVHRALELTPVDFGIT 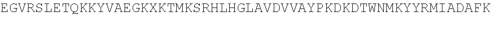 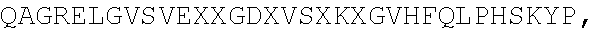
wherein X is independently selected from the following amino acids: A, R, N, D, C, Q, E, G, H, I, L, K, M, F, P, S, T, W, Y, and V and wherein at least one X is not the same amino acid as that in the same position in SEQ ID NO: 1.

4. The peptide according to claim 1 comprising a sequence according to the following:
(SEQ ID NO: 4) 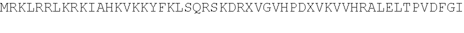
TEGVRSLETQKKYVAEGKSKTMKSRHLHGLAVDVVAYPKDKDTWNMKYYRMIADAF 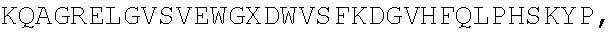
wherein X is independently selected from the following amino acids: A, R, N, D, C, Q, E, G, H, I, L, K, M, F, P, S, T, W, Y, and V and wherein at least one X is not the same amino acid as that in the same position in SEQ ID NO: 1.

5. The peptide according to claim 1 comprising a sequence according to the following:
(SEQ ID NO: 5) 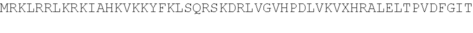
EGVRSLETQKKYVAEGKSKTMKSRHLHGLAVDVVAYPKDKDTWNMKYYRMIADAFK 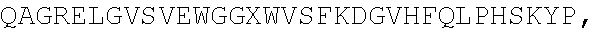
wherein X is independently selected from the following amino acids: A, R, N, D, C, Q, E, G, H, I, L, K, M, F, P, S, T, W, Y, and V and wherein at least one X is not the same amino acid as that in the same position in SEQ ID NO: 1.

6. The peptide according to claim 1 comprising a sequence according to the following:
(SEQ ID NO: 6) 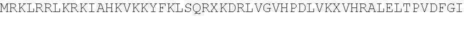 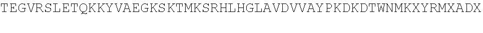
KQAGRELGVSVEWGGDWVSFKDGVHFQLPHSKYP,
wherein X is independently selected from the following amino acids: A, R, N, D, C, Q, E, G, H, I, L, K, M, F, P, S, T, W, Y, and V and wherein at least one X is not the same amino acid as that in the same position in SEQ ID NO: 1.

7. The peptide according to claim 1 comprising a sequence according to the following:
(SEQ ID NO: 7) 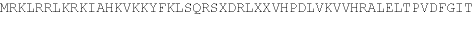
EGVRSLETQKKYVAEGKSKTMKSRHLHGLAVDVVAYPKDKDTWNMKYYRMIADAFK 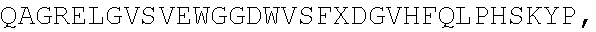
wherein X is independently selected from the following amino acids: A, R, N, D, C, Q, E, G, H, I, L, K, M, F, P, S, T, W, Y, and V and wherein at least one X is not the same amino acid as that in the same position in SEQ ID NO: 1.

8. The peptide according to claim 2, wherein when the wild-type residue of SEQ ID NO: 1 that corresponds to an X is amino acid L, then the amino acid at the X is an amino acid selected from G, A, V, I, P, F, M, and W;
wherein when the wild-type residue of SEQ ID NO: 1 that corresponds to an X is amino acid V, then the amino acid at the X is an amino acid selected from G, A, L, I, P, F, M, and W;
wherein when the wild-type residue of SEQ ID NO: 1 that corresponds to an X is amino acid W, then the amino acid at the X is an amino acid selected from G, A, V, L, I, P, F, and M;
wherein when the wild-type residue of SEQ ID NO: 1 that corresponds to an X is amino acid G, then the amino acid at the X is an amino acid selected from A, V, L, I, P, F, M, and W;
wherein when the wild-type residue of SEQ ID NO: 1 that corresponds to an X is amino acid A, then the amino acid at the X is an amino acid selected from G, V, L, I, P, F, M, and W;
wherein when the wild-type residue of SEQ ID NO: 1 that corresponds to an X is amino acid I, then the amino acid at the X is an amino acid selected from G, A, V, L, P, F, M, and W;
wherein when the wild-type residue of SEQ ID NO: 1 that corresponds to an X is amino acid F, then the amino acid at the X is an amino acid selected from G, A, V, L, I, P, M, and W;
wherein when the wild-type residue of SEQ ID NO: 1 that corresponds to an is amino acid S, then the amino acid at the X is an amino acid selected from C, T, Y, N, and Q; and
wherein when the wild-type residue of SEQ ID NO: 1 that corresponds to an X is amino acid Y, then the amino acid at the X is an amino acid selected from S, C, T, N, and Q.

9. The peptide according to claim 2, wherein when the wild-type residue of SEQ ID NO: 1 that corresponds to an X is amino acid D, then the amino acid at the X is amino acid E;
wherein when the wild-type residue of SEQ ID NO: 1 that corresponds to an X is amino acid K, then the amino acid at the X is an amino acid selected from R and H;
wherein when the wild-type residue of SEQ ID NO: 1 that corresponds to an X is amino acid R, then the amino acid at the X is an amino acid selected from K and H;
wherein when the wild-type residue of SEQ ID NO: 1 that corresponds to an X is amino acid F, then the amino acid at the X is an amino acid selected from W and Y;
wherein when the wild-type residue of SEQ ID NO: 1 that corresponds to an is amino acid W, then the amino acid at the X is an amino acid selected from F and Y;
wherein when the wild-type residue of SEQ ID NO: 1 that corresponds to an X is amino acid Y, then the amino acid at the X is an amino acid selected from F and W; and
wherein when the wild-type residue of SEQ ID NO: 1 that corresponds to an X is amino acid S, then the amino acid at the X is amino acid T.

10.-23. (canceled)

24. The peptide according to claim 1, further comprising RGLRRLGRKIAHGVKKYGPTVLRIIRIAG (SEQ ID NO: 8) attached at the N-terminal or C-terminal end of the peptide through an amine bond.

25. A peptide having an amino acid sequence selected from SEQ ID Nos: 10-56 or wherein the amino acid is SEQ ID NO: 11.

26. A fusion protein comprising one or more peptides according to claim 1 and a spore crust protein selected from CotV, CotW, CotX, CotY, CotZ, and CgeA, wherein the one or more peptides are N-terminally attached, C-terminally attached, or both N-terminally and C-terminally attached to the spore crust protein.

27. A method of inhibiting microbial growth comprising applying a growth-inhibiting amount of any one of the peptides according to claim 1 to an object, wherein the microbial growth comprises growth of Gram negative bacteria and optionally, Gram positive bacteria or wherein the microbial growth comprises growth of at least E. coli and/or Salmonella.

28.-34. (canceled)

35. A method of treating a subject for a microbial infection comprising administering to a subject in need thereof a therapeutic amount of a peptide according to claim 1 to the subject.

36. (canceled)

37. A method of preventing or reducing the incidence of a microbial infection in a subject comprising administering to one or more subjects in need thereof a therapeutic amount of a peptide according to claim 1 to the one or more subjects.

38. (canceled)

39. A spore having a peptide selected from a wt LysECD7 peptide, a wt LysECD7-SMAP29 peptide, or a peptide according to claim 1 attached to the surface or wherein the spore is modified to express the peptide on the surface of the spore, wherein the spore comprises one or more recombinant biological barcodes and wherein the spore has a genome modified to render inoperable one or more genes that are needed for spore germination and/or production of a essential metabolite or wherein the isolated spores are non-germinating and/or auxotrophic.

40.-50. (canceled)

51. A formulation comprising a peptide according to claim 1.

52. (canceled)

53. A polynucleotide encoding a peptide according to claim 1 or a peptide having an amino acid sequence selected from SEQ ID Nos: 10-56.

54. (canceled)

55. A cell modified to express the peptide according to claim 1.

56. A method of detecting a polynucleotide according to claim 53 in a composition, comprising contacting the polynucleotide with a reference sequence that is configured to hybridize with at least portion of the polynucleotide, wherein the portion hybridizes with the portion of the sequence that encodes amino acid residues 25-40, 68-73, 97-110, 119-131, or a combination thereof of the encoded LysECD7 variant peptide.

57. A vector comprising a polynucleotide according to claim 53.

58. The vector of claim 56, further comprising a promoter selected from P_{ylb}, P_{cotYZ}, P_{cotVWX}, and P_{cotX}.

59.-60. (canceled)
